(19) United States
(12) Patent Application Publication
(10) Pub. No.: US 2016/0361272 A1
(43) Pub. Date: Dec. 15, 2016

(54) EDIBLE COMPOSITION INCLUDING SAFRANAL, CROCIN, PICROCROCIN AND A VITAMIN B COMPLEX FOR TREATING THE INITIAL PHASE OF DEPRESSION

(71) Applicant: OTX SOLUTIONS, Grass (LU)

(72) Inventor: Constant THOMAOGLOU, Monaco (MC)

(73) Assignee: OTX SOLUTIONS, Grass (LU)

(21) Appl. No.: 15/120,838

(22) PCT Filed: Feb. 23, 2015
(86) PCT No.: PCT/EP2015/000404

§ 371 Date: Aug. 23, 2016

(30) Foreign Application Priority Data
Feb. 24, 2014 (FR) 14/00468

(51) Int. Cl. A61K 31/11; A61K 31/51; A61K 31/525; A61K 31/455; A61K 9/00; A61K 31/047; A61K 31/519; A61K 31/7032; A61K 31/7034; A61K 31/714; A61K 31/675

(52) U.S. Cl. CPC A61K 31/11; A61K 31/714; A61K 31/51; A61K 31/525; A61K 31/455; A61K 31/675; A61K 31/047; A61K 31/519; A61K 31/7032; A61K 31/7034; A61K 9/0053

(57) ABSTRACT

An edible composition includes safranal, crocin, picrocrocin, and a vitamin B complex including the vitamins B₁, B₂, B₃, B₆, B₈, B₉ and B₁₂; which composition is advantageously intended for the treatment of onset phase of the depressive syndrome or the prevention of the state phase of the depressive syndrome in a subject.

The present international application claims the priority of the French patent application FR 14/00468 filed on 24 Feb. 2014, the content of which is incorporated into the present patent application by reference.

### FIELD OF THE INVENTION

The present invention relates to the treatment of the depressive syndrome and focuses more specifically on a composition for treatment of the onset phase of the depressive syndrome or to the prevention of the state phase of the depressive syndrome.

### PRIOR ART

The depressive syndrome is a mood disorder dominated by sadness and a psychomotor retardation of the patient. The prevalence of this pathology is 5% of the general population. This prevalence of depression, psychiatric pathology is the most frequent in the world affecting 120 million people. Therefore, depression represents the 1^{st} cause of disability in the world.

The diagnosis of the disease is clinical and based on highlighting the elements of the depressive syndrome of which there are 3 in number:
Sad mood: moral pain, sadness, anhedonia (loss of sensation of pleasure), aboulia (loss of envy), athymhormia (loss of vital momentum), painful hyperthymia
Psychomotor retardation: bradypsychia, attention deficit, bradyphemia (slow speech), asthenia, apragmatism (loss of initiative)
Psychosomatic impact: instinct function disorders and disorder of the thought content.

To consider the depression, it is possible to distinguish 3 types:
The depression which is part of the bipolar disorder: before any depressive syndrome, it is indeed necessary to search for a history of manic episode. In this case, the treatment is totally different, and it will associate with the antidepressant treatment a mood stabilizer.
The typical form of depression or “major depressive episode” (MDE): corresponds to the complete form of the depressive syndrome which is different from a depressive syndrome minor. The diagnosis of a major depressive episode however does not prejudice the severity of the depressive episode. The diagnostic criteria DSM-IV for this pathology are the following:
   Breakdown of the previous state with more than 5 symptoms for more than 14 days
   not meeting the criteria of a mixed episode
   alteration of social functioning and/or professional
   absence of organic or toxic cause
   absence of grief <6 months
The melancholic depression: it is the most worrying form of depressive syndromes. This will require almost systematically hospitalization, because of a real suicidal risk. The diagnostic criteria DSM-IV-TR for this form are the following:
   At least 1 element among generalized anhedonia or non-reactivity to pleasurable stimuli (emotional anaesthesia)
   At least 3 of the following:
      Marked depressed mood
      Morning predominance
      Early morning awakening
      Anorexia or weight loss
      excessive or inappropriate guilt
   Beyond these criteria to define the “classic” melancholic depression, in addition we can cite specific forms of melancholic depression that are the melancholic depression stuporous, melancholic depression delusional, Cotard's syndrome (melancholic depression delusional of the elderly), and the melancholic depression anxious.

For each of these 3 types, this same depressive syndrome may have different clinical forms in function of its evolution. Thus, we can observe:
the recurring depressive disorder (unipolar disorder), which is defined by the emergence of two major depressive episodes separated by a free interval greater than 2 months.
the chronic depression which is defined by the presence of a characterized major depressive episode during at least 2 years (without free interval)
The seasonal disorder of the mood: with an onset of symptoms in the autumn and disappearance in the spring

To consider the major depressive episode (MDE) more specifically, it is necessary to also define it in relation to its severity. Thus, there are 3 distinct stages that we can define in the following manner:
MDE lighter: presence of symptoms just sufficient to diagnose (5), with little overall impact
MDE medium: more symptoms than necessary, but moderate impact on the daily life of the patient
MDE severe: almost all the symptoms with a major social impact

Off scratch, we observe on average a mean resolution from 6 months to 1 year for the major depressive episodes. However, and in the susceptibilities and predisposition functions of each patient, there may be complications linked to this MDE. As an example we can cite:
suicide: It is crucial to assess and prevent the risk of suicide in any patient presenting an MDE
scalable: the passage to the chronicity, the appearance of bipolar disorder and especially the risk of recidivism which represents 50% at the 1^{st} episode and can reach 90% during the 3^{rd} MDE
psychiatric: anxiety disorders, psychotic, or the onset of addictions (alcohol/drugs)
somatics: the constitution of bedsores (if clinophilia), the onset of denutrition (if anorexic), etc.
iatrogenics: the secondary effects of the tricyclics and serotonin reuptake inhibitors (SSRIs), the lifting of inhibition, as well as the risk of turning maniac
social: the isolation, the loss of employment, family breakdown, etc.

Under treatment, the evolution is different with remission in a shorter timeframe, with a complete remission at the end of 4 months of treatment, on average. Now, the recurrence of symptoms can occur at an early stage and before 4 months of treatment. This recurrence qualifies as a relapse. When symptoms reappear beyond 4 months of treatment, it will be recidivism.

Finally, and even under treatment, the passage to the chronicity is not excluded. Also, it will then be possible to observe persistent symptoms beyond 2 years.

There are also forms of depression resistant to treatment, these are defined by the failure of 2 treatments (including an imipramine) well conducted during 6 weeks. It should also be noted that the appearance of a manic syndrome is not excluded under treatment that is what is called “turning”.

Now, and for what is the depressive episode, this one is brought on gradually. Thus, and before the onset of symptoms required for diagnosis of the disease according to the DSM-IV criteria, it is possible to observe for the referral physician, of the environment, a clear difference with the previous condition of the subject. The first clear symptoms are often a sense of inability and decline, a growing indecision, a future deterioration. This phase is called the beginning phase of a depressive syndrome.

Then, the depressive episode settles in and we observe the symptoms afore mentioned which constitute the state phase of a depressive syndrome.

The compositions such as described in the prior art are intended only for the treatment of the state phase of the depressive syndrome and more generally the MDE, those that are mild, medium or severe.

### DETAILED DESCRIPTION OF THE INVENTION

The inventors have been able to highlight that the oral administration in some subjects, capsules with a composition comprising everything simultaneous safranal, crocin, picrocrocin and a vitamin B complex specifically already allowing at the end of a few days, to reduce all or in part of the symptoms, to obtain a significant improvement of the symptomatology of the beginning phase of a depressive syndrome. So, it is highly likely that the administration of this composition, due to its protective properties, has prevented these patients from entering the state phase of depressive syndrome.

Accordingly, a first object of the invention relates to an edible composition comprising:
safranal;
crocin;
picrocrocin; and
a vitamin B complex comprising the vitamins B₁, B₂, B₃, B₆, B₈, B₉ and B₁₂.

Vitamin B₁, corresponding to thiamin, is a water-soluble vitamin . It is a metabolic precursor of thiamin pyrophosphate (TPP), a coenzyme essential for some decarboxylase. This vitamin is essential to the transformation of carbohydrates into energy by the Krebs cycle and is necessary for the proper functioning of the nervous system and muscles.

Advantageously the composition according to the invention includes a proportion in vitamin B1 corresponding To at least 50% of the RDA, preferably at least 75% of the RDA and particularly preferred at least 100% of the RDA for vitamin B₁.

We mean by Recommended Daily Allowance, the RDA recommended in the Directive 2008/100/EC, which are 1.1 mg for vitamin B₁.

Preferably, the composition according to the invention presents a proportion in vitamin B₁ between 0.6 and 2 mg, for example between 1 and 1.5 mg, and particularly preferred of 1.1 mg.

Vitamin B₂ Is it an essential cofactor of glutathione reductase which is a key enzyme for the detoxification of the body. It is particularly used in the treatment of the substance of the migraine.

The Recommended Daily Allowance (RDA) for vitamin B₂ is 1.4 mg according to Directive 2008/100/CE.

Preferably, the composition according to the invention presents in vitamin B₂ a proportion between 1 and 2.5 mg, for example between 1.2 and 1.6 mg, and particularly preferred of 1.4 mg.

Vitamin B₃, the precursor to the NAD⁺) (nicotinamide adenine dinucleotide) and the NADP⁺ (nicotinamide adenine dinucleotide phosphate), is necessary as a cofactor of redox in the metabolism of carbohydrates, of lipids and some proteins. These different functions are an essential element of cellular metabolism.

The Recommended Daily Allowance (RDA) for vitamin B₃ is 16 mg according to Directive 2008/100/CE.

Preferably, the composition according to the invention presents in vitamin B₃ a proportion between 10 and 25 mg, for example between 13 and 19 mg, and particularly preferred of 16 mg.

Regarding the vitamin B₆, it intervenes in the metabolism of homocysteine in which the deficiency resulted in a significant decline in the tonicity. Linked with the nervous system, vitamin B₆ is also involved in the biosynthesis of serotonin.

The Recommended Daily Allowance (RDA) for vitamin B₆ is 1.4 mg according to Directive 2008/100/CE.

Preferably, the composition according to the invention presents a proportion in vitamin B₆ between 1 and 2.5 mg, for example between 1.2 and 1.6 mg, and particularly preferred of 1.4 mg.

Recent studies suggest that vitamins B₉ and B₁₂ are essential to limit the cognitive decline and onset level of age-related pathologies like Alzheimer's disease. In respect of the vitamin B₁₂ more specifically, it is essential for the normal functioning of the central and peripheral nervous system where it participates in the synthesis of neurotransmitters and the maintenance of the myelin sheath that protects the nerves and optimizes their functioning and the formation of blood.

The Recommended Daily Allowance (RDA) for vitamin B₁₂ is 2.5 μg according to Directive 2008/100/CE.

Preferably, the composition according to the invention presents a proportion in vitamin B₁₂ between 1 and 5 μg, for example between 2 and 3 μg, and particularly preferred of 2.5 μg.

The Recommended Daily Allowance (RDA) for vitamin B₉ is 200 μg according to Directive 2008/100/CE.

Preferably, the composition according to the invention presents a proportion in vitamin B₉ between 100 and 500 μg, for example between 150 and 250 μg, and particularly preferred of 200 μg.

Regarding the vitamin, B₈ is involved in the biosynthesis of vitamins B₉ and B₁₂.

The Recommended Daily Allowance (RDA) for vitamin B₈ is 55 μg according to Directive 2008/100/CE.

Preferably, the composition according to the invention presents a proportion in vitamin B_{g} between 10 and 100 μg, for example between 25 and 80 μg, and particularly preferred of 80 μg.

Advantageously again, the vitamin B complex also includes additionally the vitamin B₅.

Vitamin B5 (pantothenic acid) contributes to normal intellectual performance.

The Vitamin B5, corresponding to the pantothenic acid, or panthenol, is a water-soluble vitamin. It is a metabolic precursor of the coenzyme A, which is essential to the synthesis and metabolism of proteins, carbohydrates and lipids , but which also acts on the nervous system and the adrenal glands, where it is nicknamed “antistress vitamin”.

The Recommended Daily Allowance (RDA) for vitamin B₅ is 6 mg according to Directive 2008/100/CE.

Preferably, the composition according to the invention presents a proportion in vitamin B₈ between 1 and 12 mg, for example between 2 and 8 mg, and particularly preferred of 6 mg.

By safranal is meant 2,6,6-trimethyl-1,3-cyclohexadiene-1-carboxaldehyde of following formula:

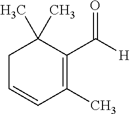

Safranal is an effective anticonvulsant which acts as an agonist of GABA_{A} receptors.

Advantageously the composition according to the invention includes Safranal in a proportion between 0.3 and 1.7 mg, preferably between 0.35 and 1mg, and particularly preferred of 0.7 mg.

By picrocrocine is meant 4-(β-D-glucopyranosyloxy)-2,6,6-trimethyl-1-cyclohexene-1-carboxaldehydede following formula:

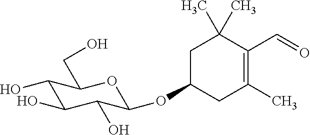 [SMILES: CC1=C(C=O)C(C)(C)C[C@H](OC2OC(CO)C(O)C(O)C2O)C1]

Advantageously the composition according to the invention includes picrocrocin, in a proportion between 1.5 and 6 MG, preferably between 2 and 4 mg, and particularly preferred of 2.8 mg.

By crocin is meant 8.8-diapo-8,8-carotenoid α-crocin digentiobiose crocetin ester acid of following formula

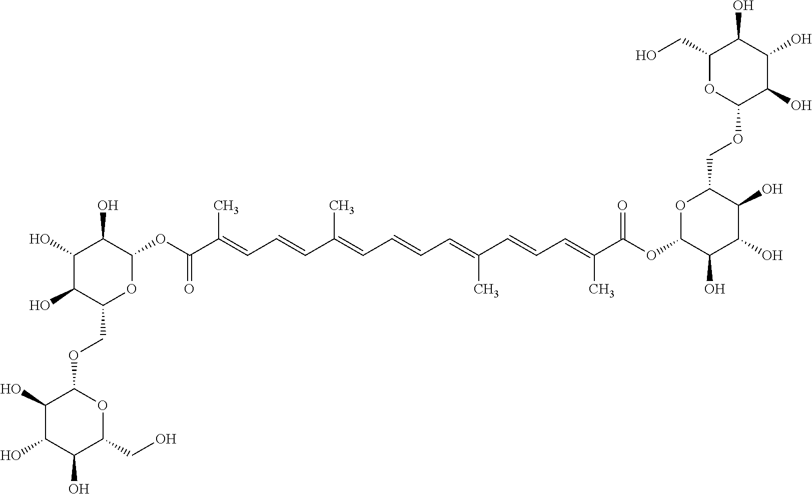 [SMILES: CC(/C=C/C=C(\C)C(=O)O[C@@H]1O[C@H](CO[C@@H]2O[C@H](CO)[C@@H](O)[C@H](O)[C@H]2O)[C@@H](O)[C@H](O)[C@H]1O)=C\C=C\C=C(C)\C=C\C=C(/C)C(=O)O[C@@H]1O[C@H](CO[C@@H]2O[C@H](CO)[C@@H](O)[C@H](O)[C@H]2O)[C@@H](O)[C@H](O)[C@H]1O]

Advantageously, the composition according to the invention includes crocin in a proportion between 0.5 and 3 mg, preferably between 1 and 2 mg, and particularly preferred of 1.4 mg.

It should be noted that the different data values for the proportions of the ingredients of the composition according to the invention refer to values with an uncertainty of 10% (±).

In combination with crocin, picrocrocin and safranal, it is to be noted that these are present in the Saffron and that they can be isolated thereof.

The saffron is derived from the culture of a flower of the species Crocus sativus, which belongs to the family of Iridaceae. This flower has the characteristic to possess three stigmas (distal ends of carpels of the plant).

The saffron, which was for several decades the most expensive spice in the world, originating in the Middle East, but was first cultivated in the Greek provinces , for more than 35 centuries. Crocus sativus grows preferentially on clay-limestone soils, which are well-watered and drained, and which also have a high content of organic matter. Traditionally, one uses it for culture of elevated beds favoring good drainage and facilitating its harvesting. The budding is at the beginning of the Autumn, but it is only in the middle that the plant begins to flower. As soon as flowering, the harvesting of the flowers must then be very quickly hand picked, which justifies the substantial cost of this spice. Additionally the saffron flourishes in a narrow window of one to two weeks, after their blooming at dawn the flowers wilt rapidly during the day. In addition to the difficulty of the crop, the stigma must be rapidly dried in order to prevent their decomposition. To do this, according to the traditional method, the stigmas are first separated on screens with fine mesh which are then placed above coal or wood burning in an open fireplace where the temperature reached 30 and 35 ° C. for 10 to 12 hours. After which the dry spice is preferably placed in an airtight glass container.

With regard to the spice, strictly speaking and as an idea, it should be in the order of 150 flowers to obtain from only the stigma harvested, 1 g of dry saffron. To produce 12 g of saffron dried (72 g before drying), therefore it must be nearly 1 kg of flowers. Also, and on average, one flower freshly cut will provide 0.03 g of saffron fresh, or 0.007 g of saffron dry.

In addition to these culinary properties, saffron has been associated very interestingly with medical properties such as appetite suppressant (gout et al., Nutrition Research, Vol. 30, p: 305-313, 2010), or anti-depressants (MOSHIRI et al., Phytomedicine, Vol.13, P:607-611, 2006).

As well, the patent application FR 2 900053 describes a composition comprising of saffron for the treatment of overweight and the patent application FR 2 975 007 describes a composition comprising of saffron with a view to stimulate sexual tonicity.

According to another particular embodiment, the composition according to the invention is in the form of a capsule, preferably a vegetable capsule.

As such a vegetable capsule in particular can be simply achieved, particularly with cellulose—based envelope (Hydroxypropylmethylcellulose, or “HPMC”, or even “Hypromellose”); to which cellulose can be supplemented with natural dye, so that the envelope has the relevant desired properties.

Preferably, the said capsule presents a green or yellow color.

Said envelope may in addition also include opacifying agents such as titanium oxide.

Other pharmaceutical agents and/or acceptable foodstuff may be added, such as antioxidant agents, bulking agents, fluidizers, natural extracts, minerals, trace elements, amino acids, fatty acids, anti-caking agents, natural oils, aromas, colorings, acidifiers, thickeners, preservatives and sweeteners.

Examples of such antioxidant agents. For examples, we are able to cite, the polyphenols, particularly the form of plant extracts (extracts of green tea, grape, ginseng), vitamin C, particularly the form of plant extracts (extract of acerola, pomegranate, citrus fruits), or even vitamin E, particularly in the form of plant extracts; or their derivatives.

Examples of bulking agents, we are able to cite microcrystalline cellulose, the potato maltodextrine or the magnesium lactate.

Examples of fluidizers, we are able to cite the magnesium silicate, magnesium stearate as well as colloidal silica.

Examples of natural extracts, we are able to cite the extracts of green tea, cinnamon, guarana, maté, fennel, meadowsweet, corn, sage, lemon balm or even coffee.

Examples of minerals or trace elements, we are able to cite magnesium, iodine, iron, copper, zinc, selenium, chromium, molybdenum, manganese, silicon, vanadium, nickel or tin.

Examples of amino acids, we are able to cite alanine, cysteine, aspartic acid, glutamic acid, phenylalanine, glycine, histidine, isoleucine, lysine, leucine, methionine, asparagine, proline, glutamine, arginine, serine, threonine, valine, tryptophan or tyrosine.

Examples of fatty acids, we are able to cite the unsaturated fatty acids such as omega-3 or omega-6.

Examples of anti-caking agents customarily used in the food industry, we are able to cite the magnesium stearate and colloidal silica.

An example of a thickener, we are able to cite the potato starch, hydroxypropyl methylcellulose, citrus pectin, guar gum, carob bean, agar-agar, konjac, hydrogenated oils or beeswax.

Example of acidifiers, we cite the citric acid.

Examples of sweeteners, we are able to cite among others, xylitol, aspartame, glucose syrup, fructooligosaccharide syrup, maltitol in powder or syrup, acesulfame potassium, fructooligosaccharide and sodium cyclamate.

Examples of preservatives, we are able to cite potassium sorbate, sodium benzoate or ascorbyl palmitate (antioxidant).

A more advantageous embodiment of said composition includes additionally at least one appropriate excipient.

All these compounds are by no way of limitation pharmaceutical agents and acceptable foodstuffs that can be added to the composition according to the invention and other agents can be envisaged.

A second objective of the invention relates to the use of a composition as previously described for the treatment of the onset phase of the depressive syndrome or to the prevention of the state phase of the depressive syndrome in a subject.

By subject, one means a human: a man or a woman.

By onset phase of the depressive syndrome, one means a subject with the following symptoms:

1) a sense of inability and depreciation linked to asthenia (tiredness),

2) growing indecision (compared to the previous state of the patient) with concentration difficulties, and

3) a future deterioration with increasing pessimism.

For example, such an onset phase of depressive syndrome can materialize in the form of a burn-out.

By state phase of a depressive syndrome, we mean highlighting of the following 3 symptoms:
Sad mood with moral pain, sadness, anhedonia, aboulia, athymhormia, and/or painful hyperthymia
Psychomotor retardation with bradypsychia, attention deficit, bradyphemia, asthenia, and/or apragmatism
Psychosomatic consequence with disorder of instinctive functions and/or disorder of the thought content

The following examples are provided to illustrate the invention and should in no case be considered as a limit to the scope of the invention.

1) Preparation of Capsules

We prepare capsules of hydroxypropylmethylcellullose (HPMC) including the composition described in the table below.

| Ingredients | Proportion | |
|---|---|---|
| Safranal | 0.6 to 0.8 | mg |
| Crocin | 1.3 to 1.5 | mg |
| Picrocrocin | 2.7 to 2.9 | mg |
| Vitamin B₁ | 1 to 1.2 | mg |
| Vitamin B₂ | 1.3 to 1.5 | mg |
| Vitamin B₃ | 15 to 17 | mg |
| Vitamin B₆ | 1.3 to 1.5 | mg |
| Vitamin B₈ | 70 to 90 | μg |
| Vitamin B₉ | 180 to 220 | μg |
| Vitamin B₁₂ | 2 to 3 | μg |

2) Evaluation of the Effectiveness on the Onset Phase of a Depressive Syndrome

The effectiveness of the composition according to the invention has been tested on a panel of several dozens of people at the beginning of the autumn period. Among the panel of people, it appears that a significant number of them presented the characteristic symptoms of those of the onset phase of a depressive syndrome.

The panel of people have ingested daily a capsule of the composition according to the invention.

It was felt a net improvement of the tonicity amongst all the panel of people, with particularly a net decrease of the symptomatology among people with symptoms of onset phase of a depressive syndrome.

This test shows the surprising effect of the composition according to the invention on the treatment of the onset phase of a depressive syndrome and, by ricochet, its effect on the prevention of the state phase of a depressive syndrome.

1. An edible composition comprising:
safranal;
crocin;
picrocrocin; and
A Vitamin B complex comprising the vitamins B₁, B₂, B₃, B6, B8, B9 and B12.

2. The composition according to claim 1, comprising a proportion of:
safranal between 0.3 and 1.7 mg, preferably between 0.35 and 1 mg, and particularly preferred of 0.7 mg;
crocin between 0.5 and 3 mg, preferably between 1 and 2 mg, and particularly preferred of 1.4 mg.
picrocrocin between 1.5 and 6 MG, preferably between 2 and 4 mg, and particularly preferred of 2.8 mg; and
a proportion of vitamin B₁ corresponding to at least 50% of the Recommended Daily Allowance (RDA),
a proportion of vitamin B₂ corresponding to at least 50% of the Recommended Daily Allowance (RDA),
a proportion of vitamin B₃ corresponding to at least 50% of the Recommended Daily Allowance (RDA),
a proportion of vitamin B₆ corresponding to at least 50% of the Recommended Daily Allowance (RDA),
a proportion of vitamin B₈ corresponding to at least 50% of the Recommended Daily Allowance (RDA),
a proportion of vitamin B₉ corresponding to at least 50% of the Recommended Daily Allowance (RDA), and
A proportion of vitamin B₁₂ corresponding to at least 50% of the Recommended Daily Allowance (RDA).

3. The composition of claim 1, which includes:
a proportion of vitamin B₁ between 0.6 and 2 mg, for example between 1 and 1.5 mg, and preferably 1.1 mg;
a proportion of vitamin B₂ between 1 and 2.5 mg, for example between 1.2 and 1.6 mg, and particularly preferred of 1.4 mg;
a proportion of vitamin B₃ between 10 and 25 mg, for example between 13 and 19 mg, and particularly preferred of 16 mg;
a proportion in vitamin B₆ between 1 and 2.5 mg, for example between 1.2 and 1.6 mg, and particularly preferred of 1.4 mg;
a proportion in vitamin B₈ between 10 and 100 μg, for example between 25 and 80 μg, and particularly preferred of 80 μg;
a proportion in vitamin B₉ between 100 and 500 μg, for example between 150 and 250 μg, and particularly preferred of 200 μg; and
a proportion of vitamin B₁₂ between 1 and 5 ηg, for example between 2 and 3 μg, and particularly preferred of 2.5 μg.

4. The composition of claim 1 presented in the form of a capsule.

5. The composition according to claim 4, wherein said capsule is in the form of a cellulose based envelope.

6. The composition of claim 1, further comprising at least a pharmaceutical agent and/or acceptable foodstuff chosen in the group comprising the antioxidant agents, bulking agents, fluidizers, natural extracts, minerals, trace elements, amino acids, fatty acids, anti-caking agents, natural oils, flavorings, colorings, acidifiers, thickeners, preservatives and sweeteners.

7. The composition according to claim 6, further comprising at least an appropriate excipient.

8. A composition according to claim 1 intended for the treatment of the onset phase of the depressive syndrome or to the prevention of the state phase of the depressive syndrome in a subject.

9. The composition according to claim 8, wherein said subject is a human.

10. The composition according to claim 8, wherein the onset phase of the depressive syndrome of a subject is defined by a subject presenting the following symptoms:
i) a sense of inability and decline linked to asthenia,
ii) increasing indecision with concentration difficulties, and
iii) future deterioration with increasing pessimism.
